(19) United States
(12) Patent Application Publication
(10) Pub. No.: US 2026/0356923 A1
(43) Pub. Date: Nov. 12, 2026

(54) sdAb-CBM FUSION PROTEIN BOUND TO POLYSACCHARIDE POLYMER

(71) Applicant: BACTOLIFE A/S, COPENHAGEN Ø (DK)

(72) Inventors: MADS LAUSTSEN, COPENHAGEN Ø (DK); SIGNE CHRISTENSEN, COPENHAGEN Ø (DK); SANDRA WINGAARD THRANE, COPENHAGEN Ø (DK); ÁGNES DUZS, COPENHAGEN Ø (DK); CHRISTEL JØRGENSEN, COPENHAGEN Ø (DK); ANDREAS HOUGAARD LAUSTSEN-KIEL, COPENHAGEN Ø (DK)

(21) Appl. No.: 19/120,362

(22) PCT Filed: Oct. 11, 2023
(86) PCT No.: PCT/EP2023/078242

§ 371 Date: Apr. 11, 2025

(30) Foreign Application Priority Data
Oct. 14, 2022 (EP) 22201628.9

(51) Int. Cl. C07K 16/1282; A61K 39/00; A61K 47/61; A61P 31/04

(52) U.S. Cl. CPC C07K 16/1282; A61K 47/61; A61P 31/04; A61K 2039/545; C07K 2317/569; C07K 2319/01

(57) ABSTRACT

A composition comprising a fusion protein comprising a single domain antibody (sdAb) and a carbohydrate-binding module (CBM) bound to a polysaccharide polymer via a not covalent binding of the polysaccharide polymer to the CBM (termed “sdAb-CBM:pSac”)—and use of it in the prevention or treatment of a pathogen induced infection in the gastrointestinal tract.

### FIELD OF THE INVENTION

The present invention relates to a composition comprising a fusion protein comprising a single domain antibody (sdAb) and a carbohydrate-binding module (CBM) bound to a polysaccharide polymer via a not covalent binding of the polysaccharide polymer to the CBM (termed “sdAb-CBM:pSac”)—and use of it in the prevention or treatment of a pathogen induced infection in the gastrointestinal tract.

### BACKGROUND OF THE INVENTION

As known in the art—a nanobody or single domain antibody (sdAb) refers to the smallest antigen binding fragment or single variable domain (“VHH”) derived from a naturally occurring heavy chain antibody.

Such single domain antibodies can be derived from antibodies raised in Camelidae species, for example in camel, llama, dromedary, alpaca and guanaco. Single domain antibodies may also be synthetically produced, such as by recombinant expression in a suitable production host cell (e.g. a bacteria, a fungal or mammalian host cell).

The molecular weight of single domain antibodies (sdAbs) is generally much smaller than common antibodies, which are composed of two heavy protein chains and two light chains.

Nanobodies have been shown to be just as specific as antibodies, and in some cases, they are more robust. The smaller size and single domain make these antibodies easier to transform into microorganism (e.g. bacteria) cells for bulk production.

WO2020/144164A1 (Bactolife) describes use of sdAbs for the treatment of a pathogen induced infection in the gastrointestinal (GI) tract of an animal (e.g. a pig, a cattle, etc) or human subject.

Use of sdAb monomers as such and divalent sdAb-linker-sdAb constructs are described—in e.g. Example 13 is shown that divalent sdAb-linker-sdAb constructs may work better than corresponding sdAb monomers.

As known in the art—a pathogen induced infection may be caused by the pathogen itself (e.g. a bacteria such as e.g. E. coli) or be caused by a molecule (e.g. a toxin) produced by the pathogen (such as e.g. toxin B produced by the pathogen Clostridium difficile).

As known in the art—carbohydrate-binding modules (CBMs) are small components of several enzymes, which present an independent fold and function, and specific carbohydrate-binding activity.

Numerous different CBMs are known, and the review paper of Oliveira et al. (Biotechnology Advances 33 (2015) 358-369) provides an overview of different known applications of recombinant CBM-fusion technology—which e.g. includes modification of fibers used in the paper and textile industry, affinity purification tools and immobilization of recombinant proteins (e.g. to a carbohydrate based hydrogel).

As discussed in the above-mentioned Oliveira review article—originally, the CBMs were classified as cellulose-binding domains (CBDs), because the first examples of these classes of protein domains bound to crystalline cellulose.

The article of Yang et al. (Applied and Environmental Microbiology, April 2020 Volume 86 Issue 8 e02938-19, p 1-15) describes use of a cellulose-binding domain for improved production of nanobodies in Bacillus subtilis bacteria host cells.

The article of Hussack et al. (Sensors 2009, 9, 5351-53) describes an immobilization platform for sensing of pathogens, biomarkers and environmental pollutants—which comprises sdAb attached to CBM and linked to a cellulose support.

The article Barbosa et al. (Acta Biomaterialia 143 (2022) 216-232) describes peptide antimicrobial cellulose-based materials (e.g. hydrogels, paper) for applications such as wound dressings, tissue scaffolds, coatings.

Without being limited to theory—at the filing date of the present patent application—the present inventors were not aware of a single prior art document that directly and unambiguously describes:
use of a fusion protein comprising a single domain antibody (sdAb) and a carbohydrate-binding module (CBM) bound to a polysaccharide polymer via a not covalent binding of the polysaccharide polymer to the CBM (termed “sdAb-CBM:pSac”) for the prevention/treatment of a pathogen induced infection in the gastrointestinal tract of an animal/human subject.

### SUMMARY OF THE INVENTION

A problem to be solved by the present invention relates to the provision of a novel pharmaceutical/dietary composition/combination that advantageously may e.g. be used for the prevention/treatment of a pathogen induced infection in the gastrointestinal tract (GI) of an animal (e.g. a pig, a cattle, etc) or human subject—such as e.g. reducing virulence in the GI and thereby stabilizing the healthy microbiome.

As discussed in further detail herein—the solution is based on that the present inventors identified several surprising advantageous properties for a composition comprising a compound, which is a fusion protein comprising a single domain antibody (sdAb) and a carbohydrate-binding module (CBM) bound to a polysaccharide polymer via a not covalent binding of the polysaccharide polymer to the CBM (termed “sdAb-CBM:pSac”) and wherein the composition is:
(i): comprising from 10³ to 10²⁰ of individual sdAb-CBM:pSac compounds per gram (g) weight of the composition as such.

The “-” in “sdAb-CBM” refers to a covalent binding—something that skilled person understands relates to that it is a fusion protein.

The “:” in “CBM:pSac” refers to a not covalent binding—as known in the art CBM binds to carbohydrate (e.g. a polysaccharide polymer) via not covalent binding.

FIG. 1 herein shows an illustrative example of a sdAb-CBM:pSac (termed sdAb-CBM:polymer in the figure) compound compared to WO2020/144164A1 (Bactolife) prior art divalent sdAb-linker-sdAb constructs.

In the non-limiting illustrative example of FIG. 1—the sdAb binds to a toxin molecule produced by the pathogen.

Below are briefly discussed some of the herein relevant technical results of working examples herein.

### Example 1

The results of this example show a surprisingly high improved sdAb resistance against gastric and intestinal proteases for the sdAb-CBM:pSac compound—this is of course advantageous for the treatment of a pathogen induced infection in the gastrointestinal tract of an animal/human.

### Example 2

The results of this example show a surprisingly high/strong binding of sdAb-CBM fusion protein to cellulose at pH 1.2—i.e. sdAb-CBM:pSac compound may function at this low pH—i.e. it may function at the gastric/stomach acid fluid with a pH between 1 and 3.

As known in the art—CBMs are small components of several enzymes—i.e. the skilled person would prima facie expect they work best at optimal activity pH values of the relevant natural enzymes.

In line of this reads above discussed review paper of Oliveira et al. on p363: “CBMs bind to cellulose at a moderately wide pH range, from 3.5 to 9.5”—i.e. the demonstrated surprisingly high/good binding of sdAb-CBM fusion protein to cellulose at pH 1.2 was a surprise to the present inventors.

### Example 3

The results of this example essentially demonstrate that it is possible in a relatively cheap/rapid way to get a composition comprising sdAb-CBM:pSac compounds with very little (if any measurable) amount of DNA contamination.

This may be an advantage for e.g. a dietary composition—since regulatory requirement in e.g. the European Union (EU) are getting more strict on e.g. DNA contamination—i.e. a relatively cheap/rapid way to remove such DNA contamination may be commercially very useful.

### Example 4

The results of this example show binding of sdAb-CBM to cellulose does not interrupt binding of antigen—this is evidently important for use of sdAb-CBM:pSac for the treatment of a pathogen induced infection as discussed herein.

### Example 5

The results of this example demonstrate a surprisingly good capacity of sdAb-CBM:pSac compounds of the invention to remove bacteria at even at low bacteria concentration—which evidently is of significant clinical relevance—since it e.g. gives the possibility to intervene early in the course of a bacterial infection (e.g. for prevention and/or prophylaxis related treatment), where the bacteria concentration generally is low.

Without being limited to theory—it is believed that the results of e.g. the working examples herein make it plausible that a composition comprising numerous individual sdAb-CBM:pSac compounds would be suitable for use in the treatment of a pathogen induced infection in the gastrointestinal tract of an animal/human subject.

Without being limited to theory—it is believed that the same would plausible also apply to other binding proteins of possible interest—such as e.g. FAB or DARPins.

Without being limited to theory—an advantage of the sdAb-CBM:pSac compound as discussed herein could be, that it as a compound as such may be able to bind a higher number of pathogens/toxins as compared to e.g. a sdAb-linker-sdAb construct of the prior art—something that may be an advantage for removal of pathogens/toxins present in low amounts in the gastrointestinal tract (see e.g. Example 5 herein).

As discussed in WO2020/144164A1 (Bactolife)—crosslinking of pathogens may be important for removal pathogens—e.g. to create a bigger structure that relatively easily may be removed from the gastrointestinal tract.

Without being limited to theory—an advantage of the sdAb-CBM:pSac compound could be that less crosslinking of pathogens/toxins may be required for e.g. proper clearance/removal, since the sdAb-CBM:pSac compound is by itself relatively big and may therefore relatively easily be removed from the gastrointestinal tract—something that e.g. could be an advantage if pathogens/toxins are present in low amounts and it may thereby be statistically difficult to crosslink sufficient pathogens/toxins in order to get a sufficient big molecule that relatively easily may be removed from the gastrointestinal tract.

If crosslinking should be relevant for removal of a particular pathogens/toxins—sdAb-CBM:pSac compound as discussed herein may comprise numerous sdAb bindings sites—i.e. may be very useful for relatively rapidly get crosslinking of many pathogens/toxins.

Accordingly, a first aspect of the present invention relates to a pharmaceutical or dietary composition comprising a compound, which is a fusion protein comprising a single domain antibody (sdAb) and a carbohydrate-binding module (CBM) bound to a polysaccharide polymer via a not covalent binding of the polysaccharide polymer to the CBM (termed “sdAb-CBM:pSac”) and wherein the composition is: (i): comprising from 10³ to 10²⁰ of individual sdAb-CBM:pSac compounds per gram (g) weight of the composition as such;
and wherein the sdAb binds at least one pathogen surface component and/or at least one molecule produced by a pathogen.

The term “individual” in relation to the term “individual sdAb-CBM:pSac compounds” of the first aspect should be understood as the skilled person would understand it in the present context—i.e. that it refers to that there is no significant binding between the individual sdAb-CBM:pSac compounds.

Accordingly, after the composition of the first aspect is administered enterally (i.e. via the gastrointestinal tract) the individual sdAb-CBM:pSac compounds of the composition may then actively work at different places of the gastric system and thereby remove pathogens/toxins at different places of the gastrointestinal (GI) tract—i.e. thereby obtain treatment of a pathogen induced infection at different places of the gastrointestinal tract.

In line with above—individual sdAb-CBM:pSac compounds does not have significant crosslinking to a polymer of another individual sdAb-CBM:pSac compound of the composition.

According to the art—the term “pathogen” relates to any organism or agent that can produce a disease—wherein the term disease is understood broadly to cover e.g. a pathogen induced infection in the gastrointestinal tract of an animal or human subject—i.e. the disease is preferably related to a pathogen induced infection in the gastrointestinal tract of an animal or human subject.

As discussed above—the review paper of Oliveira et al. and the article of Yang et al. discuss different known applications of recombinant CBM-fusion technology—which e.g. includes modification of fibers used in the paper and textile industry, affinity purification tools and immobilization of recombinant proteins (e.g. to a carbohydrate based hydrogel).

As understood by the skilled person—none of these prior art articles directly and unambiguously describes a pharmaceutical/dietary composition of the first aspect—for instance due to that CBM based immobilization of paper/textile and a hydrogel evidently does not give a composition comprising numerous individual sdAb-CBM:pSac compounds as required in the first aspect.

Further—skilled person understand that CBM/CBD based immobilization on a purification column is not related to a pharmaceutical or dietary composition of the first aspect.

As known in the art, CBM may bind to a polysaccharide polymer with a relatively high binding efficiency—accordingly, the sdAb-CBM and polysaccharide polymer may be administered to an animal or human subject of interest as a pharmaceutical combination for e.g. separate or sequential medical use.

For instance—if sdAb-CBM fusion proteins and polysaccharide polymer are administered as two separate compositions, then it is believed that enough sdAb-CBM proteins and polysaccharide polymers would be able to find each other in the gastrointestinal tract in order to in vivo create sufficient number of sdAb-CBM:pSac compounds to get a herein relevant prevention/treatment of a pathogen induced infection in the gastrointestinal tract of an animal or human subject.

As understood by the skilled person in the present context—it may be preferred that a pharmaceutical or dietary composition of the first aspect is administered to an animal or human subject.

Accordingly, a second aspect of the present invention relates to pharmaceutical or dietary combination comprising:
(A): a pharmaceutical or dietary composition comprising from 10³ to 10²⁰ of individual fusion proteins per gram (g) weight of the composition as such, wherein the fusion proteins are comprising a single domain antibody (sdAb) and a carbohydrate-binding module (CBM) (termed “sdAb-CBM”), and wherein the sdAb binds at least one pathogen surface component and/or at least one molecule produced by a pathogen;
and
(B): a pharmaceutical or dietary composition comprising polysaccharide polymers, wherein the polysaccharide polymers are capable of binding to the CBM of (A) via a not covalent binding of the polysaccharide polymer to the CBM in order to create individual sdAb-CBM:pSac compounds;
   for the simultaneous, separate, or sequential use in the prevention or treatment of a pathogen induced infection in the gastrointestinal tract of an animal or human subject.

As understood by the skilled person in the present context—in relation to the herein discussed combination treatment it is not essential if (A) and (B) are administrated e.g. simultaneous as a single composition or e.g. sequentially as two separate compositions. The important matter is that an effective amount of the compound/agent first administered is in in the gastrointestinal tract of the animal/human subject when the second compound/agent is administered.

Accordingly, the term “combination” of the second aspect relates herein to the various combinations of (A) and (B), for example in a single pharmaceutical/dietary composition or in separate compositions. The order of applying (A) and (B) is not essential.

A combination of (A) and (B) can be formulated for its simultaneous, separate or sequential administration. Preferably, if the administration is not simultaneous, the compositions (A) and (B) are administered in a relatively close time proximity to each other.

As known in the art—a pathogen induced infection may be caused by the pathogen itself (e.g. a bacteria such as e.g. E. coli) or be caused by molecule (e.g. a toxin) produced by the pathogen (such as e.g. toxin B produced by the pathogen Clostridium difficile).

As understood by the skilled person—the term “prevention or treatment of a pathogen induced infection in the gastrointestinal tract” should be understood as the skilled person would understand it in the present context—i.e. to broadly cover any herein relevant prevention/treatment of a pathogen induced infection—such as e.g. removal, clearance, blocking and/or inactivating the pathogens/toxins or e.g. reducing risk of infection in the sense that the pathogens do not colonize in the gastric system or e.g. reducing virulence in the GI and thereby stabilizing the healthy microbiome or e.g. so-called microbiome engineering, where e.g. number of unwanted (e.g. due to possible involvement in a disease) pathogens (e.g. E. coli bacteria) are reduced—for instance could the number of possible relevant E. coli bacteria be reduced, if these are suspected to be involved in the development of a relevant disease (such as e.g. inflammatory bowel disease (IBD) or such as e.g. an unpleasant condition due to an unbalanced microbiome).

Embodiments of the present invention are described below, by way of examples only.

### DRAWING DESCRIPTION

FIG. 1: Illustration of an example of sdAb-CBM:pSac compound as discussed herein compared to WO2020/144164A1 (Bactolife) prior art divalent sdAb-linker-sdAb constructs.

FIG. 2: Gel discussed in working example 3 herein.

### DETAILED DESCRIPTION OF THE INVENTION

### Pharmaceutical or Dietary Composition:

The skilled person knows if a composition of interest is pharmaceutical or dietary composition or not—i.e. it comprises pharmaceutical or dietary acceptable components.

The pharmaceutical or dietary composition may comprise different types of sdAb, CBM and/or pSac.

For instance—the composition may comprise different types of sdAbs binding to e.g. different pathogens/toxins of interest.

For instance—the composition may comprise some individual sdAb-CBM:pSac compounds, where pSac is e.g. a cellulose polymer and other individual sdAb-CBM:pSac compounds, where pSac is e.g. a starch polymer.

As known in the art—the pharmaceutical or dietary composition may be coated—for instance with an enteric coating to prevent dissolution or disintegration in the gastric environment, which may be preferred it main target of the composition is the intestine.

For a pharmaceutical composition, all components (alternatively termed excipients) of the composition should be pharmaceutically acceptable. By “pharmaceutically acceptable” we mean a non-toxic material that does not significantly decrease the effectiveness of the sbAb. Such pharmaceutically acceptable buffers, carriers or excipients are well-known in the art (see Remington's Pharmaceutical Sciences, 18th edition, A.R Gennaro, Ed., Mack Publishing Company (1990) and handbook of Pharmaceutical Excipients, 3rd edition, A. Kibbe, Ed., Pharmaceutical Press (2000)).

The pharmaceutical composition may further comprise one or more further active components such as e.g. an antibiotics, fecal matter transfer and/or monoclonal antibodies.

The pharmaceutical composition may e.g. be a tablet, a gel, a suspension, a solution, a liposome, a capsule, a powder, or encapsulated in polymers—preferably, the pharmaceutical composition is a tablet, a capsule or a powder.

For a dietary composition, all components (alternatively termed excipients) of the composition should be dietarily acceptable.

In one embodiment, the dietary composition comprises one or more of prebiotics, probiotics, synbiotics, proteins, lipids, carbohydrates, vitamins, fibers, and/or nutrients, such as dietary minerals.

In one embodiment, the dietary composition is a food, a feed, or a beverage—or a food additive, a feed additive, or a beverage additive—or food ingredient, a feed ingredient, or a beverage ingredient—or food supplement, a feed supplement, or a beverage supplement.

The dietary composition may e.g. be a pellet (preferably when the dietary composition is a feed), tablet, a gel, a suspension, a solution, a liposome, a capsule, a beverage, a powder, or encapsulated in polymers—preferably, the dietary composition is a pellet (preferably when the dietary composition is a feed), tablet, a capsule, a beverage or a powder.

When the subject is an animal and the dietary composition is a feed—then it is most preferred that the dietary composition is a pellet composition.

When the subject is a human—then it is most preferred that the dietary composition is a tablet or a powder—which can e.g. be used as a beverage supplement.

The dietary composition may e.g. be a so-called Medical Devise composition—such as e.g. a Medical Devise composition in accordance with e.g. European Union (EU) Medical Devices Regulation (MDR) 2017/745—for instance a so-called class II medical devise according to e.g. the MDR.

The dietary composition may e.g. be a so-called medical food composition.

For commercial relevant use—it may be preferred the pharmaceutical or dietary composition of the first aspect and/or embodiment thereof, is a composition with a weight of the composition as such of at least 100 g, more preferably least 200 g, even more preferably least 500 g, and most preferably at least 1 kg.

### Fusion Protein

Knowing the relevant sequences of sdAb and CBM of interest—it is routine work the skilled person to obtain a herein suitable sdAb-CBM fusion protein by use a e.g. relevant recombinant expression technology—i.e. it is herein not considered necessary to discuss this in great details.

As discussed above—the “-” in “sdAb-CBM” refers to a covalent binding—something that skilled person understands relates to it is a fusion protein.

As known in the art—it may be relevant to have a suitable linker sequence between the sdAb and CBM—i.e. the sdAb-CBM covalent binding does not have to be a direct covalent binding between sdAb and CBM.

The preferred linker is a glycine serine linker of the structure (GₓS)ₙ, where x may be a number between 1 to 50, preferably between 1 to 25, more preferably between 1 to 10, most preferably between 2 to 5, and n refers to a number of repeats of the GxS sequence, where n may be between 1 to 50, preferably between 1 to 25, more preferably between 1 to 10, most preferably between 2 to 5.

The sdAb-CBM fusion protein may comprises more than one sdAb—such as e.g. two or more sdAbs—which may be different sdAbs binding to e.g. different pathogens/toxins.

The fusion protein may also comprise other elements of interest—such as e.g. polypeptide/protein sequences that e.g. could facilitate purification.

The discussed sdAb-CBM fusion proteins of working examples herein are all examples of sdAb-CBM fusion proteins, where sdAb and CBM are bound together via covalent peptide bonds.

Preferably, the sdAb-CBM fusion protein of the first and/or second aspect and/or embodiment thereof is a sdAb-CBM fusion protein, wherein the sdAb-CBM fusion protein is a polypeptide sequence (preferably recombinantly expressed) and wherein the sdAb and CBM are bound together via covalent peptide bonds—e.g. directly or e.g. via a suitable amino acid linker sequence between the sdAb and CBM.

Single Domain Antibody (sdAb)

For the skilled person it is routine work to obtain a suitable sdAb in relation to a suitable pathogen of interest—i.e. it is herein not considered necessary to discuss this in great details.

As discussed above, and as known in the art—a nanobody or single domain antibody (sdAb) refers to the smallest antigen binding fragment or single variable domain (“VHH”) derived from a naturally occurring heavy chain antibody.

Such single domain antibodies can be derived from antibodies raised in e.g. sharks or e.g. Camelidae species, for example in camel, llama, dromedary, alpaca and guanaco. Single domain antibodies may also be synthetically produced, such as by recombinant expression in a suitable production host cell (e.g. a bacteria, a fungal or mammalian hoist cell).

Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies.

The term single domain antibody, in its broadest sense, is not limited to a specific biological source or to a specific method of preparation. For example, the single domain antibodies of the disclosure can generally be obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, and in particular from a mammalian species, such as from a human being, or by expression of a nucleic acid encoding such a camelized VH domain; (5) by “camelization” of a “domain antibody” or “Dab,” as described in the art, or by expression of a nucleic acid encoding such a camelized VH domain; (6) by using synthetic or semi-synthetic techniques for preparing proteins, polypeptides or other amino acid sequences known per se: (7) by preparing a nucleic acid encoding a single domain antibody using techniques for nucleic acid synthesis known per se, followed by expression of the nucleic acid thus obtained; and/or (8) by any combination of one or more of the foregoing.

As known in the art—a sdAb may comprise relevant complementary-determining regions—i.e. in a preferred embodiment, the sdAb is comprising:
(i): a complementary-determining region 1 (CDR1) comprising or consisting of a relevant sequence (may e.g. be termed SEQ ID NO: i);
(ii): a complementary-determining region 2 (CDR2) comprising or consisting of a relevant sequence (may e.g. be termed SEQ ID NO: ii); and
(iii): a complementary-determining region 3 (CDR3) comprising or consisting of a relevant sequence (may e.g. be termed SEQ ID NO: iii).

### Carbohydrate-Binding Module (CBM)

As discussed above, and as known in the art—carbohydrate-binding modules (CBMs) are small components of several enzymes, which present an independent fold and function, and specific carbohydrate-binding activity.

Numerous different CBMs are known—se e.g. above discussed review paper of Oliveira et al.

As discussed in the above-mentioned Oliveira review article—originally, the CBMs were classified as cellulose-binding domains (CBDs), because the first examples of these classes of protein domains bound to crystalline cellulose.

Accordingly, the skilled person knows or may routinely determine—if protein of interest comprises a CBM—i.e. may routinely determine if a fusion protein of interest is a sdAb-CBM fusion protein or not.

Herein cited Oliveira review article describes that CBMs can be divided into different so-called fold families (se e.g. Table 1 of the Oliveira review article)—i.e. the skilled person may based on e.g. the herein cited Oliveira review article and relevant common general knowledge routinely determine whether a CBM of interest belongs to a specific fold family (e.g. fold family 3) or not.

Preferably, the CBM belongs to any of the fold families 1-3—more preferably the CBM belongs to any of the fold families 1 or 3—even more preferably the CBM belongs to the fold family 3.

As understood by the skilled person—the preferred choice of a specific CBM relates to the preferred polysaccharide polymer of interest (see below)—i.e. if the polysaccharide polymer of interest is a cellulose polymer then shall the suitable CBM be able to bind the cellulose polymer.

If the polysaccharide polymer is a cellulose polymer—a preferred CBM may be a type A CBM.

As discussed in herein cited Oliveira review article—type A CBM or surface-binding CBM have a flat hydrophobic binding surface comprised of aromatic residues—i.e. the skilled person may based on e.g. the herein cited Oliveira review article and relevant common general knowledge routinely determine whether a CBM of interest is a type A CBM or not.

In working examples herein were used CBM comprising SEQ ID NO: 3 or SEQ ID NO: 4—accordingly, it may be preferred that the CBM is comprising SEQ ID NO: 3 or SEQ ID NO: 4.

### Polysaccharide Polymer

As known in the art—polysaccharides (alternatively termed polycarbohydrates) are chain polymeric carbohydrates composed of monosaccharide units bound together by glycosidic linkages.

The polysaccharide polymer may be linear or branched.

The polysaccharide polymer may be a homopolysaccharide or a heteropolysaccharide.

According to the art—some of the monosaccharide monomers may be modified and the skilled person would objectively still consider it a polysaccharide polymer—the skilled person may based on common general knowledge routinely determine whether polymer of interest is a polysaccharide polymer or not.

The polysaccharide polymer may comprise not monosaccharide structures/elements and still be understood to be as herein suitable polysaccharide polymer.

Relevant aspect herein e.g. reads: “carbohydrate-binding module (CBM) bound to a polysaccharide polymer via a not covalent binding of the polysaccharide polymer to the CBM”—i.e. if the polysaccharide polymer comprise not monosaccharide structures/elements then is the CBM of course bound to the polysaccharide polymer part of the polymer as such.

In working examples herein were e.g. used a polysaccharide cellulose polymer comprising around 100 to 400 glucose monomers.

The skilled person may routinely determine the preferred numbers of monomers in a polysaccharide polymer of interest—but preferably it should at least longer than 9 monomers.

Preferably, the polysaccharide polymer comprises from 10 to 50000 monomers, more preferably from 10 to 10000 monomers, even more preferably from 10 to 8000 monomers, and most preferably from 50 to 1000 monomers.

Preferably, the polysaccharide polymer is a cellulose polymer, a derivative of a cellulose polymer, a hemicellulose polymer, a derivative of a hemicellulose polymer, a starch polymer, or a derivative of a starch polymer.

As known in the art—in chemistry, a derivative is a compound that is derived from a similar compound by a chemical reaction.

As known in the art—chitosan may be considered as a derivative of chitin and both chitosan and chitin are polysaccharide polymers.

Accordingly, the skilled person knows what is understood by a derivative of a polysaccharide polymer of interest (e.g. a derivative of a cellulose polymer)—the skilled person may based on common general knowledge routinely determine whether a polysaccharide polymer of interest is a herein relevant derivative or not—such as e.g. a derivative of a cellulose polymer.

In a preferred embodiment, the polysaccharide polymer is a cellulose polymer, a hemicellulose polymer, or a starch polymer—more preferably a cellulose polymer or a hemicellulose polymer—even more preferably a cellulose polymer.

A preferred example of a hemicellulose polymer is an arabinoxylan polymer.

It may be preferred that the polysaccharide polymer is plant-based/derived polysaccharide.

The polysaccharide polymer (e.g. preferably cellulose polymer) may be a crystalline or amorphous polymer—it may be preferred that the polysaccharide polymer (e.g. preferably cellulose polymer) is an amorphous polymer.

In working examples herein were used microcrystalline cellulose (MCC) polymers.

Accordingly, it may be that the polysaccharide polymer (e.g. preferably cellulose polymer) is an crystalline polymer—such as preferably a microcrystalline cellulose (MCC) polymer.

It may be particular preferred that the polysaccharide polymer is a crystalline cellulose polymer (preferably MCC) if the CBM belongs to any of the fold families 1 or 3 (even more preferably if the CBM belongs to the fold family 3).

sdAb-CBM:pSac Compounds

Based on common general knowledge and technical teaching herein—the skilled person may routinely obtain herein a relevant sdAb-CBM:pSac compound of interest.

For instance—after e.g. recombinant expression of sdAb-CBM fusion protein—the polysaccharide polymer may be added to the cell lysate (or supernatant) comprising the expressed sdAb-CBM fusion protein and the sdAb-CBM:pSac compounds may then be created/obtained by not covalent binding of the polysaccharide polymer to the CBM.

The sdAb-CBM:pSac compounds may be obtained in relatively pure form by e.g. simple centrifugation or more sophisticated known purification technology.

As discussed above in relation to e.g. a pharmaceutical/dietary combination of the second aspect—if sdAb-CBM fusion proteins and polysaccharide polymer are administered as two separate compositions then it is believed that enough sdAb-CBM proteins and polysaccharide polymers would be able to find each other in the gastrointestinal tract in order to in vivo create sufficient of sdAb-CBM:pSac compounds to get a herein relevant the prevention/treatment of a pathogen induced infection in the gastrointestinal tract of an animal or human subject.

As discussed above, the first aspect relates in point (i) to that the composition is comprising from 10³ to 10²⁰ of individual sdAb-CBM:pSac compounds per gram (g) weight of the composition as such.

Preferably, the composition is comprising from 10⁵ to 10²⁰ of individual sdAb-CBM:pSac compounds per gram (g) weight of the composition as such, more preferably the composition is comprising from 10⁵ to 10¹⁹ of individual sdAb-CBM:pSac compounds per gram (g) weight of the composition as such, even more preferably the composition is comprising from 10⁶ to 10¹⁹ of individual sdAb-CBM:pSac compounds per gram (g) weight of the composition as such; and most preferably the composition is comprising from 10¹⁰ to 10¹⁸ of individual sdAb-CBM:pSac compounds per gram (g) weight of the composition as such.

As discussed above—the composition of a herein relevant aspect may e.g. be suspension/solution. As known in the art—1 ml of an aqueous solution has a weight of close to 1 mg.

The composition of a herein relevant aspect may comprise different types of sdAb-CBM:pSac compounds—for instance with different types of sdAbs directed to different types of pathogen.

For instance, the composition of the first aspect may comprise 10⁵/g of sdAb-CBM:pSac compounds with sdAb directed to (i.e. binding) E. coli bacteria pathogens and also 10⁵/g of sdAb-CBM:pSac different compounds with sdAb directed to (i.e. binding) another bacteria pathogens—such as composition then comprises 2×10⁵/g sdAb-CBM:pSac compounds.

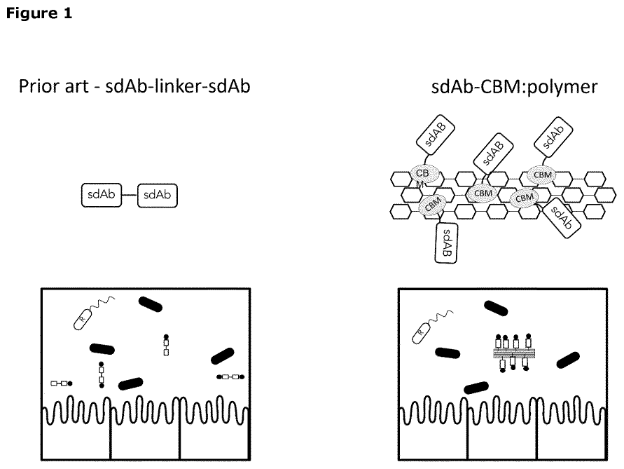

A preferred sdAb-CBM:pSac compound may e.g. be wherein the compound comprises more than one sdAb-CBM bound to the polysaccharide polymer (see e.g. FIG. 1 for an illustrative example).

In relation to a sdAb-CBM:pSac compound, wherein the compound comprises more than one sdAb-CBM bound to the polysaccharide polymer—it may be preferred that the more than one (i.e. two or more, such as 5 or more or such as 10 or more) sdAb-CBMs bound to the polysaccharide polymer are comprising different types of sdAbs (e.g. one sdAb-CBM comprises one type of sdAb and another sdAb-CBM comprises a different type of sdAb)—accordingly the sdAb-CBM:pSac compound will comprise different types of sdAbs that e.g. could bind to different types of pathogens or bind to different epitopes of the same pathogen.

sdAb Bind Pathogen or at Least One Molecule Produced by a Pathogen

As discussed above, the herein relevant aspects relate to that the sdAb binds at least one pathogen surface component and/or at least one molecule produced by a pathogen.

As discussed above—for the skilled person it is routine work to obtain a suitable sdAb in relation to a suitable pathogen of interest—i.e. it is herein not considered necessary to discuss this in great details.

Preferably, the pathogen is an enterotoxic pathogen and/or at least one molecule (preferably an enterotoxin) produced by an enterotoxic pathogen.

As known in the art—an enterotoxin is a toxin released by a pathogen (e.g. a microorganism) that targets the intestine—i.e. an enterotoxic pathogen (preferably an enterotoxic bacterium or an enterotoxic virus) is a pathogen that targets the intestine.

Preferably, the pathogen is a bacterium, a virus, a fungus, a protozoa and/or a worm—more preferably the pathogen is a bacterium or a virus (e.g. a rotavirus or a norovirus).

Preferably, the pathogen is an Escherichia coli bacteria, a Vibrio cholera bacteria, a Salmonella, a Campylobacter bacteria, a Staphylococcus bacteria, a Listeria bacteria, a Shigella bacteria, Mycoplasma and/or Clostridium difficile.

Preferably, the molecule produced by a pathogen is a toxin, an inhibitor and/or an enzyme, such as betalactamase—more preferably, the molecule produced by a pathogen is a toxin.

Preferably, the toxin is toxin B produced by the pathogen Clostridium difficile—see e.g. EP21211996.0 (Bactolife—not published at filing/priority date of present application—does e.g. not mention anything of relevance in relation to e.g. CBM).

In working examples herein were used a sdAb comprising SEQ ID NO: 1—which may be a preferred sdAb.

The sdAb comprising SEQ ID NO: 1 binds to a toxin produced by the pathogen Clostridium difficile.

### Pharmaceutical/Dietary Composition—Other Matter

As discussed above—the results of Example 3 herein essentially demonstrate that it is possible in a relatively cheap/rapid way to get a composition comprising sdAb-CBM:pSac compounds with very little (if any measurable) amount of DNA contamination.

In short—the method to remove DNA may be seen as based on that the inventors found that surprisingly little (if any measurable) amount of DNA binds to sdAb-CBM:pSac compounds, and these sdAb-CBM:pSac compounds relatively easy precipitate in a liquid culture medium used for e.g. recombinant expression based production.

This may be an advantage for e.g. a dietary composition—since regulatory requirement in e.g. the European Union (EU) are getting more strict on e.g. DNA contamination—i.e. a relatively cheap/rapid way to remove such DNA contamination may be commercially very useful.

Preferably, the composition of first aspect (preferably wherein the composition is a dietary composition) comprises less than 15 ng DNA per gram (g) weight of the composition as such—more preferably, the composition comprises less than 10 ng DNA per gram (g) weight of the composition as such—most preferably, the composition comprises less than 5 ng DNA per gram (g) weight of the composition as such.

The European Food Safety Authority (EFSA) requires no detection of DNA by a method with a detection limit of 10 ng or below (DOI: 0.2903/j.efsa.2021.6851).

### Use in Prevention or Treatment of a Pathogen Induced Infection

As discussed above, the second aspect relates to pharmaceutical or dietary combination comprising:
(A): a pharmaceutical or dietary composition comprising from 10³ to 10²⁰ of individual fusion proteins per gram (g) weight of the composition as such, wherein the fusion proteins are comprising a single domain antibody (sdAb) and a carbohydrate-binding module (CBM) (termed “sdAb-CBM”), and wherein the sdAb binds at least one pathogen surface component and/or at least one molecule produced by a pathogen;
and
(B): a pharmaceutical or dietary composition comprising polysaccharide polymers, wherein the polysaccharide polymers are capable of binding to the CBM of (A) via a not covalent binding of the polysaccharide polymer to the CBM in order to create individual sdAb-CBM:pSac compounds; for the simultaneous, separate, or sequential use in the prevention or treatment of a pathogen induced infection in the gastrointestinal tract of an animal or human subject.

As understood by the skilled person—if the compositions (A) and (B) are e.g. two separate compositions—then will the preferred amount of sdAb-CBM fusion proteins in (A) generally depend on the amount polysaccharide polymers in (B)—if there is e.g. 10⁶ polysaccharide polymers in (B) then it will normally be preferred that there is at least 10⁶ sdAb-CBM fusion proteins in (A).

It may be even more preferred to have a significant higher number of sdAb-CBM fusion proteins in (A) than polysaccharide polymers in (B) to increase the possibility to get individual sdAb-CBM:pSac compounds, wherein the compounds comprises more than one sdAb-CBM bound to the polysaccharide polymer (see e.g. FIG. 1 for an illustrative example).

Just as a possible suitable example—the composition (B) comprising polysaccharide polymers (e.g. a food or feed composition) may e.g. be mixed with the composition (A) comprising “sdAb-CBM” protein before given to the animal or human subject of interest.

Preferably, the composition (A) is comprising from 10⁵ to 10²⁰ of individual sdAb-CBM per gram (g) weight of the composition as such, more preferably the composition is comprising from 10⁵ to 10¹⁹ of individual sdAb-CBM per gram (g) weight of the composition as such, even more preferably the composition is comprising from 10⁶ to 10¹⁹ of individual sdAb-CBM per gram (g) weight of the composition as such; and most preferably the composition is comprising from 10¹⁰ to 10¹⁸ of individual sdAb-CBM per gram (g) weight of the composition as such.

Preferably, the pharmaceutical combination is a single composition comprising both Compound (A) and Compound (B)—such as most preferably, wherein the second aspect relates to a pharmaceutical or dietary composition of the first aspect and/or embodiment thereof, for use in the prevention or treatment of a pathogen induced infection in the gastrointestinal tract of an animal or human subject

Preferably, the gastrointestinal tract is intestine—i.e. a prevention or treatment of a pathogen induced infection in the intestine of an animal or human subject.

As discussed above—the prior art (e.g. WO2020/144164A1 (Bactolife)) describes use of sdAbs for the treatment of a pathogen induced infection in the gastrointestinal (GI) tract of an animal (e.g. a pig, a cattle, etc) or human subject.

Accordingly, it is routine work for the skilled person to use a composition/combination as descried herein for the medical use of the second aspect—i.e. it is herein not considered necessary to discuss this in great details.

Preferably, the animal is a pig, a cattle, a poultry, a sheep, a goat, a horse, a chicken, a donkey, a mule, a duck, a geese or a turkey—more preferably the animal is a pig, a cattle or a poultry—most preferably the animal is a pig.

Preferably, the composition is administered enterally—such as orally, such as a food supplement, as a tablet, a gum (e.g. a gummy bear) or a gel, or via gastric intubation.

As known in the art—the term “enterally” relates administration via the gastrointestinal tract.

It may be preferred that the prevention or treatment is for reducing virulence in the GI and thereby stabilizing the healthy microbiome—in particular, when the composition is a dietary composition.

### a Separate Aspect of the Invention—a Method to Remove DNA

As discussed above, the inventors found that surprisingly little amounts (if any measurable) amount of DNA binds to sdAb-CBM:pSac compounds—it is believed that the same would plausible also apply to other proteins (Prot) of possible interest.

Accordingly, a separate aspect of the invention relates to a method to obtain a composition comprising a compound, which is a fusion protein comprising a protein (Prot) and a carbohydrate-binding module (CBM) bound to a polysaccharide polymer via a not covalent binding of the polysaccharide polymer to the CBM (termed “Prot-CBM:pSac”) and wherein the composition is:
(i): comprising from 10³ to 10²⁰ of individual Prot-CBM:pSac compounds;
and wherein the method is comprising following steps:
(A): recombinant expression of from 10³ to 10²⁰ of individual Prot-CBM in a suitable microorganism host cell;
(B): obtaining a sample of the expressed Prot-CBM compounds of (A), where a significant amount of the host cells have been removed;
(C): adding pSac to the sample of (B) to create a sample of from 10³ to 10²⁰ of individual Prot-CBM:pSac compounds;
(D): precipitating the Prot-CBM:pSac compounds of (C) to get a sample with liquid and precipitated Prot-CBM:pSac compounds; and
(E): removing DNA from the sample of (D) by separating (e.g. by washing) the liquid from the precipitated Prot-CBM:pSac compounds to get the composition comprising the individual Prot-CBM:pSac compounds with removed DNA.

The individual steps of the method may be considered as routine known steps for the skilled person.

For instance, if the recombinant expressed Prot-CBM compounds are secreted to the culture medium in step (A)—then may a significant amount of host cell be removed by e.g. centrifugation to get sample (B).

If expressed Prot-CBM compounds are not secreted (e.g. if E. coli is used as host cell)—cell lysis will generally be required.

The skilled person may routinely determine how much pSac is preferably added in step (C)—it will generally depend on the number of expressed Prot-CBM compounds.

Precipitating the Prot-CBM:pSac compounds in step (D) may routinely be done by e.g. centrifugation or other suitable routine techniques.

Step (E) is a routine step essentially relating to separating liquid comprising DNA from the precipitated Prot-CBM:pSac compounds—as known in the art, this may e.g. be done by washing (with e.g. water or a suitable aqueous buffer/solution).

Preferably the protein (Prot) in the DNA removal method of the separate aspect is a single domain antibody (sdAb).

Preferably, there is in step (E) removed so much DNA that one gets (e.g. after other optional relevant steps—such as relevant up-concentration steps) a composition that comprises less than 15 ng DNA per gram (g) weight of the composition as such—more preferably, the composition comprises less than 10 ng DNA per gram (g) weight of the composition as such—most preferably, the composition comprises less than 5 ng DNA per gram (g) weight of the composition as such.

An advantage of the method of the separate aspect relates to that it is not necessary to use DNase and/or column-based purification steps to remove the DNA—accordingly, a preferred embodiment of the method of the separate aspect relates to a method, wherein the method does not involve use of DNase and/or column-based purification steps.

Other herein described preferred embodiments for the composition of the first aspect (e.g. preferred CBM, preferred pSac, etc.) are also preferred embodiments for DNA removal method of the separate aspect.

### EXAMPLES

The examples below refer to below mentioned materials and methods related references—these references are as such not related to the inventive concept of the present invention.
EP21211996.0 (Bactolife—not published at filing/priority date of present application)
Martin et al (BMC Biotechnol. 2006; 6: 46)
Studier et al (Protein Expression and Purification 2005 Volume 41, Issue 1, Pages 207-234)
Ghamghami et al (AMB Expr 10, 129 (2020))
Harmsen et al (Applied Microbiology and Biotechnology 2007, volume 77 issue 1 p13-22)
Fiil et al. (iScience 25, 104003 Apr. 15, 2022)

### Example 1: Binding to Cellulose Enhances Resistance Against Gastric and Intestinal Proteases

### Materials and Methods

### Composition:

| sdAb | Linker | CBM | |
|---|---|---|---|
| B004 | QVQLQESGGGLVQAGDSLRLS | GGGGSGGG | TQSHYGQCGGIGYSGPTVCAS |
| 0005 | CLASGRTFRYYAMGWFRQAPG | GSGGGGS | GTTCQVLNPYYSQCL (SEQ ID |
| | KEREFVAGINISGSNTDYSDS | (SEQ ID | NO: 3 - uniprot ID: G0RVK1, |
| | VKGRFTISKDNAKNMGYLQMN | NO: 2) | residue 478-514) |
| | SLKPEDTAVYYCAANRRGRND | | |
| | YEYWGRGTQVTVSS | | |
| | (SEQ ID NO: 1) | | |
| B004 | SEQ ID NO: 1 | SEQ ID | CSSVWGQCGGQNWSGPTCCAS |
| 0006 | | NO: 2 | GSTCVYSNDYYSQCL |
| | | | (SEQ ID NO: 4 - Uniprot |
| | | | ID: P07987,residue 26-62) |
| B003 | SEQ ID NO: 1 | SEQ ID | |
| 0005 | | NO: 2 | |

SEQ ID NO: 1 is identical to SEQ ID NO: 27 of EP21211996.0 (Bactolife - not published at filing/priority date of present application)

sdAb: The sdAb in the sdAB-CBM fusion proteins B004_0005 and B004_0006 as well as both sdAb domains in the sdAb-sdAb fusion protein B003_0005 comprises SEQ ID NO: 1.

CBM: Two CBMs were used the CBM domain from T. reesei Cellobiohydrolase 11, cel7a (B004_0005) and T. reseei Cellobiohydrolase II, cel6a (B004_0006). See table above.

Polysaccharide polymer: Cellulose polymer Avicel pH 101—overall ranging from ~100 to 400 glucose monomers—from provider Sigma Aldrich.

Protein Production and sdAb-CBM:polymer Assembly

The sdAb-CBM and sdAb-sdAb fusion proteins was cloned into the pSANG10-3F vector (Martin, 2006) ensuring C-terminal 6×His-tag and 3×FLAG tag.

Proteins were expressed in an E. coli strain in autoinduction media (Studier, 2005). After lysis and extraction of periplasmic fraction (Ghamghami, 2020) proteins were purified using Immobilized-metal affinity chromatography (IMAC).

The sdAb-CBM:pSac compounds of the composition were assembled by incubating Avicel PH-101 and sdAb-CBM (4 μmol sdAb-CBM per 1 g of Avicel PH-101) with end-over-end rotation at room temperature for 1 hour.

### Test of Protease Resistance

Resistance towards proteases was investigated by mixing sdAb-CBM:pSac, sdAb-CBM or sdAb-sdAb with 35 mM NaCl, 0.07 HCl, 200 U/L pepsin pH 2 (simulated gastric fluid (SGF)) or 50 mM K3PO4, 10 mg/mL pancreatin pH 6.8 (simulated intestine fluid (SIF)). The degree of resistance towards the proteases was evaluated by remaining full-length protein as evaluated by SDS-PAGE.

### Results

The table below shows remaining full-length protein (in % of full length protein detected before treatment) after treatment with SGF or SIF.

| | | SGF | | | SIF | | |
|---|---|---|---|---|---|---|---|
| | Time (min) | 10 | 30 | 120 | 10 | 30 | 120 |
| B004_0005:avicel | sdAb-CBM:pSac | 48 | 27 | 2 | 35 | 15 | 3 |
| B004_0006:avicel | sdAb-CBM:pSac | 46 | 30 | 12 | 51 | 13 | 4 |
| B004_0005 | sdAb-CBM | 26 | 2 | 0 | 6 | 0 | 0 |
| B004_0006 | sdAb-CBM | 13 | 0 | 0 | 0 | 0 | 0 |
| B003_0005 + avicel | sdAb-sdAb + avicel | 0 | 0 | 0 | 15 | 1 | 0 |
| B003_0005 | sdAb-sdAb | 0 | 0 | 0 | 21 | 0.6 | 0 |

The compound sdAb-CBM:polymer is approximately 2-folds more resistant towards the gastric protease pepsin than sdAb-CBM and at least 46-fold more resistant than a sdAb-sdAb fusion after 10 min of treatment with SGF. Even more surprising, after 2 hours of exposure to a simulated gastric environment full length sdAb-CBM was still present in the sdAb-CBM:polymer composition despite all sdAb-sdAb was degraded after only 10 min as well as minimum 98% of sdAb-CBM was degraded after 30 min in a composition not comprising a carbohydrate polymer. Further, the sdAb-CBM:polymer composition exhibits ~2 fold increased resistance towards pancreatin in SIF after 10 min. And, similarly to treatment with SGF detectable levels of sdAb-CBM is present after 120 min of treatment with SIF when in a sdAb-CBM:polymer composition, whereas no full-length sdAb-CBM is detected after 30 min when not associated with a carbohydrate. Similarly, very little, if any, sdAb-sdAb is detected after 30 min treatment with SIF and none after 120 min.

The assembly of the sdAb-CBM:polymer is essential for the higher resistance towards proteases. This is evident by the observation that a composition comprising sdAb-sdAb and Avicel PH-101 does not exhibit increased resistance towards proteases.

It is well known in the art that sdAbs have high physicochemical stability as well as resistance against proteases (Harmsen, 2007, Asaadi 2021). Hence, it was highly surprising to the present inventors that the compound (sdAb-CBM:pSac) showed increased resistance to SGF and SIF both short term (10 min) and over an increased period of time (120 min).

### Conclusions

The results of this example show a surprisingly high improved sdAb resistance against gastric and intestinal proteases for the sdAb-CBM:pSac compound—this is of course advantageous for the treatment of a pathogen induced infection in the gastrointestinal tract of an animal/human.

### Example 2: The sdAb-CBM Fusion Protein Binds Cellulose at High Temperature and Low pH

### Materials and Methods

Same sdAb-CBM sequences and protein expression and lysis as in example 1.

Periplasmic fraction from lysed cells were incubated with Avicel PH-101 (1 g Avicel PH-101 per 100 mL culture) and incubated at room temperature with end-to-end rotation for 1 hour to assemble sdAb-CBM:polymer composition. sdAb-CBM:polymer was washed with 150 mM NaCl (10 mL per gram of Avicel PH-101). sdAb-CBM-polymer was treated with 10 mL HCl (pH 1.2) per gram of Avicel PH-101. Absorption of sdAb-CBM to Avicel PH-101 was evaluated by SDS-PAGE before and after treatment.

### Results

After treatment of sdAb-CBM:polymer with HCl pH 1.2-98% of sdAb-CBM remains bound to avicel pH-101. In light of previous reports of CBM binding to cellulose in pH ranging from 3.5 to 9 (Oliverira, 2015), this high degree of binding at a significant lower pH must be considered surprising.

### Conclusions

The results of this example show a surprisingly high/good binding of sdAb-CBM fusion protein to cellulose at pH 1.2—i.e. sdAb-CBM:pSac compound may function at this low pH—i.e. it may function at the gastric/stomach acid fluid with a pH between 1 and 3.

### Example 3: Binding to Cellulose Reduces DNA Contamination when Expressed in E. coli

### Materials and Methods

Same sdAb-CBM sequences, protein expression and lysis as in example 1. Periplasmic fraction from lysed cells were incubated with 80 μL Ni-NTA beads (Thermo Scientific) or 800 μg Avicel PH-101 (Sigma Aldrich) per mL of cell lysate with end-over-end rotation for 1 hour at room temperature. Soluble and insoluble (containing sdAb-CBM:avicel or sdAb-CBM:Ni-NTA) fractions were separated by centrifugation. Insoluble fraction was washed 3 times with ⅕ of the lysate volume. sdAb-CBM was eluted by 5 M urea and desalted using PD-10 column (Cytiva). Treatment with DNAse I (Applichem Lifescience) was performed with 117 U DNase I (brand) per 100 μL cell lysate, avicel-elute or Ni-NTA elute. DNA content was evaluated by gel electrophoresis on 1% agarose.

### Results

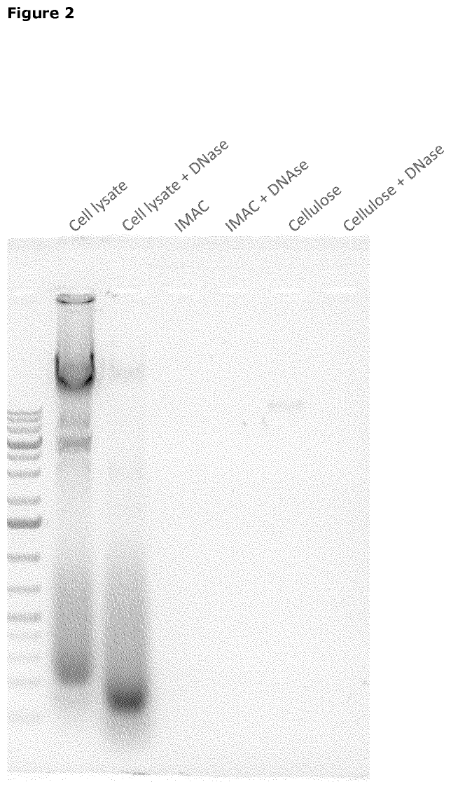

FIG. 2 shows that the DNA content in a sdAb-CBM composition expressed in E. coli is reduced significantly by binding to Avicel PH-101 compared with treatment with the nuclease DNAse I. Further, the DNA content after binding to Avicel PH-101 is similar to binding to Ni-NTA beads highly specific for His-tagged proteins.

### Conclusions

The results of this example essentially demonstrates that it is possible in a relatively cheap/rapid way to get a composition comprising sdAb-CBM:pSac compounds with very little (if any measurable) amount of DNA contamination.

### Example 4: Binding of sdAb-CBM to Cellulose does not Interrupt Binding of Antigen

### Materials and Methods

sdAb-CBM and sdAb-sdAB sequences and protein production as in example 1. Assembly of sdAb-CB:polymer as in example 1, but with 1.5 μmol sdAb-CBM per gram of avicel. Antigen (Uniprot ID: P18177.3 residue 1-543) was inserted in modified pSANG10 without FLAG-tag. Expression and purification of antigen as proteins in example 1.

sdAb-CBM:polymer, sdAb-CBM or sdAb-sdAB was incubated with antigen in 3 times molar excess (in relation to binding site) with end-over-end rotation at room temperature for 2 h. antigen:sdAb-CBM and antigen:sdAb-sdAb:antigen complex were pulled down by centrifugation by addition of anti-FLAG beads (insoluble fraction). Antigen:sdAb-CBM:polymer (insoluble fraction) complex was pulled down by centrifugation. Bound antigen (in insoluble fraction) as fraction of total antigen was evaluated by SDS-PAGE.

### Results

| | Antigen bound/total |
|---|---|
| | antigen (%) |
| B004_0006:polymer | 98 |
| B004_0006 | 76 |
| B003_0005 | 82 |
| Avicel | 12 |

Table above shows that 98% of available antigen is bound to a sdAb-CBM:polymer composition compared to 76% for sdAb-CBM not bound to a carbohydrate polymer and 82% for sdAb-sdAb. This indicates that assemble of the sdAb-CBM:polymer composition does not interfere with antigen binding. And, at best, might increase antigen binding compared to a sdAb-sdAb composition.

### Conclusions

The results of this example show binding of sdAb-CBM to cellulose does not interrupt binding of antigen—this is evidently important for use of sdAb-CBM:pSac for the treatment of a pathogen induced infection as discussed herein.

### Example 5: Comparing “sdAb-CBM:pSac” (Invention) with “sdAb-sdAb” (Prior Art)—Removal E. coli from Solution

As discussed above—WO2020/144164A1 (Bactolife) describes divalent sdAb-linker-sdAb constructs—alternatively herein termed “sdAb-sdAb fusion protein” or “sdAb-sdAb”.

In this comparative example was compared sdAb-CBM:pSac compounds of the invention with comparative (i.e. same sdAb) prior art related sdAb-sdAb.

### Materials and Methods

### Composition:

| | sdAb | Linker | CBM |
|---|---|---|---|
| B004 0011 | QVQLQESGGGLVQPGGSLRLSCLVSG | SEQ ID NO: 2 | SEQ ID NO: 4 |
| | GTFSWYAMGWFRQAPGKEREFVATV | (See Example 1). | (See Example 1 - |
| | SRGGGSSYYADSVKGRFTISRDNAKN | | cel6a) |
| | TVYLQMNSLKPEDTAVYYCAAGRGAP | | |
| | SDTGRPDEYDYWGQGTQVTVSS | | |
| | (SEQ ID NO: 5) | | |
| B004 0010 | SEQ ID NO: 5 | SEQ ID NO: 2 | SEQ ID NO: 3 |
| | | (See Example 1). | (See Example 1 - |
| | | | cel7a) |
| B001 0003 | SEQ ID NO: 5 | SEQ ID NO: 2 | |
| | | (See Example 1). | |

sdAb: The sdAb in the sdAB-CBM fusion proteins B004_0011 and B004_0010 as well as both sdAb domains in the sdAb-sdAb fusion protein B001_0003 comprises SEQ ID NO: 5.

SEQ ID NO: 5 is known in the art to be a sdAb binding to E. Coli and the Fiil et al. (iScience 25, 104003 Apr. 15, 2022) article reads: “K922 (GenBank accession number: AJ810819)”.

CBM: The same cel6a and cel7a as used in Example 1.

Polysaccharide polymer: Cellulose polymer Avicel pH 101—see Example 1.

Protein Production and sdAb-CBM:Polymer Assembly

The protein production and sdAb-CBM:polymer assembly were essentially done in accordance with Example 1 with relatively minor changes based on the common general knowledge of the skilled person.

The sdAb-CBM:pSac (Invention) compounds of the invention are termed B004_0011:Avicel and B004_0010:Avicel.

The “prior art” comparative sdAb-sdAb compound is termed B001_0003.

Test of Capacity for Removal of E. Coli from a Solution

The capacity for removal of E. Coli from a solution of B004_0011:Avicel and B004_0010:Avicel (Invention) as compared to B001_0003 (prior art) were done by incubating antigen presenting ETEC F4+ bacteria with sdAb-CBM:polymer or sdAb-sdAb followed by centrifugation and quantification of bacteria remaining in solution. Specifically, antigen presenting ETEC F4+ bacteria stock solution was adjusted to a concentration 1000 CFU per 1 ml. The bacterial stock solution was then mixed with B004_0011:Avicel or B004_0010:Avicel or B001_0003 in a 1:1 ratio with end-over-end rotation at room temperature for 1 hr.

Bacteria:sdAb-CBM:polymer or bacteria:sdAb-sdAb:bacteria complex were pulled down by centrifugation at 50 RCF for 30 seconds. Representative fraction of supernatant was cultured overnight on LB agar plates at 30 °C to quantify CFU per ml.

The used bacteria concentration is what objectively herein may be said to be a low bacteria concentration.

### Results

The tables below show the results of the test of capacity for removal of E. Coli from a solution.

Bacteria without Protein and without Polysaccharide (Avicel)

| Bacterial count | Error at 95% CI |
|---|---|
| (CFU/ml) | (CFU/ml) |
| 1133.3 | 97.4 |

Bacteria without Protein and with Polysaccharide (Avicel)

| Concentration of | | |
|---|---|---|
| poly saccharide | | |
| without protein | Bacterial count | Error at 95% CI |
| (mg/ml) | (CFU/ml) | (CFU/ml) |
| 4.0 | 1270.0 | 175.4 |

Bacteria with B004_0011:Avicel

| Concentration of | | |
|---|---|---|
| Active Fiber in | | |
| binding sites | Bacterial count | Error at 95% CI |
| (nmol/ml) | (CFU/ml) | (CFU/ml) |
| 0.07 | 796.7 | 116.8 |
| 0.69 | 730.0 | 111.6 |
| 1.44 | 570.0 | 75.3 |
| 2.39 | 643.3 | 89.5 |
| 4.79 | 533.3 | 74.2 |

Bacteria with B004_0010:Avicel

| Concentration of | | |
|---|---|---|
| Active Fiber in | | |
| binding sites | Bacterial count | Error at 95% CI |
| (nmol/ml) | (CFU/ml) | (CFU/ml) |
| 0.07 | 883.3 | 115.1 |
| 0.69 | 766.7 | 95.5 |
| 1.35 | 663.3 | 94.2 |
| 2.26 | 680.0 | 109.8 |
| 4.52 | 546.7 | 78.5 |

Bacteria with B001_0003—“Prior Art” Comparative sdAb-sdAb

| Concentration of | | |
|---|---|---|
| B001_0003 in | | |
| binding sites | Bacterial count | Error at 95% CI |
| (nmol/ml) | (CFU/ml) | (CFU/ml) |
| 0.69 | 1173.3 | 103.3 |
| 1.44 | 1183.3 | 139.4 |
| 2.39 | 1240.0 | 107.8 |
| 4.79 | 1163.3 | 104.4 |

### Conclusions

As discussed above—WO2020/144164A1 (Bactolife) describes divalent sdAb-linker-sdAb constructs—alternatively herein termed “sdAb-sdAb fusion protein” or “sdAb-sdAb”.

In this comparative example was compared sdAb-CBM:pSac compounds of the invention with comparative (i.e. same sdAb) prior art related sdAb-sdAb.

The results of this example demonstrated a surprisingly high improved capacity of removal of E. coli from solution of sdAb-CBM:pSac compounds of the invention (B004_0011:Avicel and B004_0010:Avicel) as compared to comparative (i.e. same sdAb) prior art related sdAb-sdAb (B001_0003).

This advantageous removal of bacterial effect was also significant even at low bacteria concentration—something which evidently is very advantageous/useful—e.g. for removal of pathogens/toxins present in low amounts in the gastrointestinal tract.

The fact that compounds of present invention advantageous can remove bacteria at even at low bacteria concentration gives the possibility to intervene early in the course of a bacterial infection (e.g. for prevention and/or prophylaxis related treatment), where the bacteria concentration generally is low.

### Example 6: Loading of B004_0011 and B004_0010 onto Two Different Celluloses

### Materials and Methods

The sdAB-CBM fusion proteins B004_0011 and B004_0010 of Example 5 were bound/loaded onto two different cellulose polymers—Avicel PH-101 (i.e. as in Example 5) and MCC-102 cellulose.

MCC-102 may be obtained from provider Sigma Aldrich.

Like Avicel pH 101—MCC-102 is also a cellulose polymer—both are what in the art may be termed microcrystalline cellulose (MCC).

### Results

The results (data as such not shown) demonstrated that both of sdAB-CBM fusion proteins B004_0011 and B004_0010 could bind properly to both Avicel pH 101 and MCC-102—i.e. both worked satisfactory and are thereby examples of herein suitable polymers.

### Conclusions

The results of this example show that sdAB-CBM fusion proteins may be properly bound to different polysaccharide polymers—e.g. different cellulose polymers.

### REFERENCE LIST

1: WO2020/144164A1 (Bactolife)
2: Oliveira et al. (Biotechnology Advances 33 (2015) 358-369)
3: Yang et al. (Applied and Environmental Microbiology, April 2020 Volume 86 Issue 8 e02938-19, p 1-15)
4: Hussack et al. (Sensors 2009, 9, 5351-53)
5: Barbosa et al. (Acta Biomaterialia 143 (2022) 216-232)

What is claimed is:

1. A method for the simultaneous, separate, or sequential use in the prevention or treatment of a pathogen induced infection in the gastrointestinal tract of an animal or human subject, comprising administering to the subject a therapeutically effective amount of a pharmaceutical or dietary composition comprising a compound, which is a fusion protein comprising a single domain antibody (sdAb) and a carbohydrate-binding module (CBM) bound to a polysaccharide polymer via a not covalent binding of the polysaccharide polymer to the CBM (termed “sdAb-CBM:pSac”) and wherein the composition is:
(i): comprising from 10³ to 10²⁰ of individual sdAb-CBM:pSac compounds per gram (g) weight of the composition as such;
and wherein the sdAb binds at least one pathogen surface component and/or at least one molecule produced by a pathogen.

2. The method of claim 1, wherein the composition comprises from 10¹⁰ to 10¹⁸ of individual sdAb-CBM:pSac compounds per gram (g) weight of the composition.

3. The method of claim 1, wherein the composition comprises less than 15 ng DNA per gram (g) weight of the composition.

4. The method of claim 3, wherein the composition is a dietary composition, and wherein the dietary composition is a food, a feed, or a beverage—or a food additive, a feed additive, or a beverage additive—or a food ingredient, a feed ingredient, or a beverage ingredient—or a food supplement, a feed supplement, or a beverage supplement.

5. The method of claim 1, wherein the combination is administered enterally, preferably orally, and wherein the gastrointestinal tract is intestine.

6. The method of claim 1, wherein the subject is an animal and wherein the animal is a pig, a cattle or a poultry.

7. The method of claim 1, wherein the CBM belongs to any of the fold families 1-3.

8. The method of claim 7, wherein the CBM belongs to fold family 3.

9. The method of claim 1, wherein the polysaccharide polymer comprises from 10 to 50000 monomers, more preferably from 10 to 10000 monomers, even more preferably from 10 to 8000 monomers, and most preferably from 50 to 1000 monomers.

10. The method of claim 1, wherein the polysaccharide polymer is a cellulose polymer, a hemicellulose polymer, or a starch polymer.

11. The method of claim 10, wherein the polysaccharide polymer is a cellulose polymer.

12. The method of claim 11, wherein the CBM is a type A CBM.

13. The method of claim 11, wherein the CBM comprises SEQ ID NO: 3 or SEQ ID NO: 4 and wherein the sdAb comprises SEQ ID NO: 1.

14. The method of claim 1, wherein the pathogen is an enterotoxic bacterium.

15. A method for the simultaneous, separate, or sequential use in the prevention or treatment of a pathogen induced infection in the gastrointestinal tract of an animal or human subject, comprising administering to the subject a therapeutically effective amount of a pharmaceutical or dietary combination comprising:
(A): a pharmaceutical or dietary composition comprising from 10³ to 10²⁰ of individual fusion proteins per gram (g) weight of the composition as such, wherein the fusion proteins are comprising a single domain antibody (sdAb) and a carbohydrate-binding module (CBM) (termed “sdAb-CBM”), and wherein the sdAb binds at least one pathogen surface component and/or at least one molecule produced by a pathogen;
and
(B): a pharmaceutical or dietary composition comprising polysaccharide polymers, wherein the polysaccharide polymers are capable of binding to the CBM of (A) via a not covalent binding of the polysaccharide polymer to the CBM in order to create individual sdAb-CBM:pSac compounds.


SEQUENCE LISTING (5 sequences)

SEQ ID NO: 1 - protein, 119 aa, unidentified
QVQLQESGGGLVQAGDSLRLSCLASGRTFRYYAMGWFRQAPGKEREFVAGINISGSNTDYSDSVKGRFTISKDNAKNMGYLQMNSLKPEDTAVYYCAANRRGRNDYEYWGRGTQVTVSS

SEQ ID NO: 2 - protein, 15 aa, unidentified
GGGGSGGGGSGGGGS

SEQ ID NO: 3 - protein, 36 aa, unidentified
TQSHYGQCGGIGYSGPTVCASGTTCQVLNPYYSQCL

SEQ ID NO: 4 - protein, 36 aa, unidentified
CSSVWGQCGGQNWSGPTCCASGSTCVYSNDYYSQCL

SEQ ID NO: 5 - protein, 125 aa, unidentified
QVQLQESGGGLVQPGGSLRLSCLVSGGTFSWYAMGWFRQAPGKEREFVATVSRGGGSSYYADSVKGRFTISRDNAKNTVYLQMNSLKPEDTAVYYCAAGRGAPSDTGRPDEYDYWGQGTQVTVSS